(19) United States
(12) Patent Application Publication
(10) Pub. No.: US 2023/0071885 A1
(43) Pub. Date: Mar. 9, 2023

(54) PREDICTION OF PANCREATIC DUCTAL ADENOCARCINOMA USING COMPUTED TOMOGRAPHY IMAGES OF PANCREAS

(71) Applicants: CEDARS-SINAI MEDICAL CENTER, Los Angeles, CA (US); SOUTHERN CALIFORNIA PERMANENTE MEDICAL GROUP, Los Angeles, CA (US)

(72) Inventors: Bechien Wu, La Canada Flintridge, CA (US); Srinivas Gaddam, Los Angeles, CA (US); Debiao Li, South Pasadena, CA (US); Stephen Jacob Pandol, Los Angeles, CA (US); Touseef Ahmad Qureshi, Los Angeles, CA (US)

(73) Assignees: CEDARS-SINAI MEDICAL CENTER, Los Angeles, CA (US); SOUTHERN CALIFORNIA PERMANENTE MEDICAL GROUP, Los Angeles, CA (US)

(21) Appl. No.: 17/790,942

(22) PCT Filed: Jan. 12, 2021
(86) PCT No.: PCT/US2021/013093

§ 371 Date: Jul. 5, 2022

Related U.S. Application Data
(60) Provisional application No. 62/960,405, filed on Jan. 13, 2020.

(51) Int. Cl. G16H 50/30; G16H 50/70; G06T 7/00; G06T 7/155

(52) U.S. Cl. CPC G16H 50/30; G16H 50/70; G06T 7/0014; G06T 7/155; G06T 2207/30096; G06T 2207/20081; G06T 2207/10081

(57) ABSTRACT

According to some implementations of the present disclosure, a system for identifying individuals at risk for PDAC includes a CT scanner, a memory, and a control system. The CT scanner is configured to generate CT image data associated with a pancreas of a patient. The memory stores machine-readable instructions. The control system includes one or more processors configured to execute the machine-readable instructions. The CT image data associated with the pancreas of the patient is received. The received CT image data is processed to output a set of CT image features. The set of CT image features is received as an input to a machine learning PDAC prediction algorithm. An indication of whether the patient is at high risk for PDAC is determined as an output of the machine learning PDAC prediction algorithm.

### CROSS-REFERENCE TO RELATED APPLICATIONS

This application claims priority to and the benefit of U.S. Provisional Patent Application No. 62/960,405, filed on Jan. 13, 2020, which is hereby incorporated by reference herein in its entirety.

### TECHNICAL FIELD

This disclosure relates generally to systems and methods for prediction of medical conditions, and more particularly, to systems and methods for prediction of pancreatic ductal adenocarcinoma using computed tomography images of pancreas.

### BACKGROUND

Pancreatic Ductal Adenocarcinoma (PDAC), a common type of Pancreatic Cancer, is one of the leading causes of cancer-related deaths in both men and women in the United States. The 5-year survival rate for PDAC is only about 7-8%. Typically, by the time PDAC is diagnosed for the first time, it is usually very late in the disease stage. Furthermore, managing PDAC is subject to cost, patient discomfort, and high ratio of mortality.

Therefore, it is highly needed to predict PDAC at a stage when cancer is better treatable. Moreover, with early-stage diagnosis, the 5-year survival rate can be increased to as high as 20%. Thus, identification of individuals at high risk for PDAC will have high clinical significance, as follow-up imaging examinations or biopsy on these individuals may lead to early detection and surgical intervention when the tumors are still resectable and not metastatic.

However, PDAC predication is difficult due to the lack of reliable screening tools, the absence of sensitive and specific biomarkers, and low prevalence. Thus, a need exists for an automated tool that aids in predicting the most common types of pancreatic cancer, such as PDAC. The present disclosure is directed to solving these problems and addressing other needs.

### SUMMARY

According to some implementations of the present disclosure, a system for identifying individuals at risk for PDAC includes a CT scanner, a memory, and a control system. The CT scanner is configured to generate CT image data associated with a pancreas of a patient. The memory stores machine-readable instructions. The control system includes one or more processors configured to execute the machine-readable instructions. The CT image data associated with the pancreas of the patient is received. The received CT image data is processed to output a set of CT image features. The set of CT image features is received as an input to a machine learning PDAC prediction algorithm. An indication of whether the patient is at high risk for PDAC is determined as an output of the machine learning PDAC prediction algorithm.

In some examples, the indication of whether the patient is at high risk for PDAC is displayed on a display device of the system.

In some examples, the machine learning PDAC prediction algorithm is trained with historical data for historical patients. The historical data includes a plurality of CT image features of a pancreas and a corresponding PDAC diagnosis of each of the historical patients. The plurality of CT image features is extracted from retrospective CT images of the pancreas of the each of the historical patients. In some examples, the PDAC diagnosis is healthy, pre-cancerous, or cancerous.

In some examples, the set of CT image features is indicative of a variation in morphology of the pancreas. In some examples, the morphology includes a size, a shape, a signal intensity, or any combination thereof.

In some examples, the set of CT image features is indicative of a change in texture of the pancreas. In some examples, the set of CT image features includes at least one of tissue heterogeneity, run length non-uniformity, inverse autocorrelation, long run emphasis, and short run emphasis.

In some examples, the machine learning PDAC prediction algorithm includes a K-means clustering, a Logistic Regression, a Support Vector Machine, a Naïve Bayes classifier, a Nearest Neighbors, or any combination thereof. In some examples, the machine learning PDAC prediction algorithm includes a Naïve Bayes classifier.

According to some implementations of the present disclosure, a method for identifying individuals at risk for PDAC using machine learning is disclosed. Data associated with a plurality of individuals is received. The data including historical data of historical patients and current data of a current patient. The current data includes a set of CT image features associated with CT images of a pancreas of the current patient. A machine learning algorithm is trained with the historical data, such that the machine learning algorithm is configured to (i) receive, as an input, the current data of the current patient, and (ii) determine, as an output, an indication of whether the current patient is at high risk for PDAC.

In some examples, the historical data includes retrospective CT images of a pancreas and a corresponding PDAC diagnosis of each of the historical patients. In some examples, the PDAC diagnosis is healthy, pre-cancerous, or cancerous.

In some examples, the historical data includes a plurality of CT image features of a pancreas and a corresponding PDAC diagnosis of each of the historical patients, the plurality of CT image features being extracted from retrospective CT images of the pancreas of the each of the historical patients.

In some examples, the set of CT image features associated with the CT images of the pancreas of the current patient is extracted from the CT images of the pancreas of the current patient.

In some examples, the plurality of CT image features of the historical data is indicative of a variation in morphology of the pancreas. In some examples, the morphology includes a size, a shape, a signal intensity, or any combination thereof.

In some examples, the plurality of CT image features of the historical data is indicative of a change in texture of the pancreas.

In some examples, the plurality of CT image features of the historical data includes at least one of tissue heterogeneity, run length non-uniformity, inverse autocorrelation, long run emphasis, and short run emphasis.

In some examples, the machine learning algorithm includes a K-means clustering, a Logistic Regression, a Support Vector Machine, a Naïve Bayes classifier, a Nearest Neighbors, or any combination thereof. In some examples, the machine learning algorithm includes a Naïve Bayes classifier.

According to some implementations of the present disclosure, a method for identifying individuals at risk for PDAC is disclosed. CT image data associated with a pancreas of a patient is generated using a CT scanner. The CT image data is processed, using one or more processors, to output a set of CT image features. The set of CT image features is received as an input to a PDAC prediction model. An indication of whether the patient is at high risk for PDAC is determined as an output of the PDAC prediction model. The indication is displayed on a display device.

In some examples, the determining the indication of whether the patient is at high risk for PDAC includes determining whether the set of CT image features is indicative of pre-cancerous tissue changes in the pancreas of the patient.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The foregoing and other advantages of the present disclosure will become apparent upon reading the following detailed description and upon reference to the drawings.

FIG. 1 is a functional block diagram of a system for identifying individuals at risk for PDAC, according to some implementations of the present disclosure;

FIG. 2 is a flow diagram of a method for identifying individuals at risk for PDAC, according to some implementations of the present disclosure;

FIG. 3 is a flow diagram of a method for identifying individuals at risk for PDAC using machine learning, according to some implementations of the present disclosure;

FIG. 4 is a Manhattan plot of predictive features over horizontal line at p-value=0.05, according to some implementations of the present disclosure; and

FIG. 5 is a combined feature map of five predictors for healthy pancreas on the left, and for pre-cancerous pancreas on the right, according to some implementations of the present disclosure.

While the present disclosure is susceptible to various modifications and alternative forms, specific implementations have been shown by way of example in the drawings and will be described in further detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration. It should be understood that numerous specific details, relationships, and methods are set forth to provide a full understanding of the disclosure. One having ordinary skill in the relevant art, however, will readily recognize that the disclosure can be practiced without one or more of the specific details, or with other methods. In other instances, well-known structures or operations are not shown in detail to avoid obscuring the disclosure. The present disclosure is not limited by the illustrated ordering of acts or events, as some acts may occur in different orders and/or concurrently with other acts or events. Furthermore, not all illustrated acts or events are required to implement a methodology in accordance with the present disclosure.

Aspects of the present disclosure can be implemented using one or more suitable processing device, such as general purpose computer systems. microprocessors, digital signal processors, micro-controllers, application specific integrated circuits (ASIC), programmable logic devices (PLD), field programmable logic devices (FPLD), field programmable gate arrays (FPGA), mobile devices such as a mobile telephone or personal digital assistants (PDA), a local server, a remote server, wearable computers, tablet computers, or the like.

Memory storage devices of the one or more processing devices can include a machine-readable medium on which is stored one or more sets of instructions (e.g., software) embodying any one or more of the methodologies or functions described herein. The instructions can further be transmitted or received over a network via a network transmitter receiver. While the machine-readable medium can be a single medium, the term “machine-readable medium” should be taken to include a single medium or multiple media (e.g., a centralized or distributed database, and/or associated caches and servers) that store the one or more sets of instructions. The term “machine-readable medium” can also be taken to include any medium that is capable of storing, encoding, or carrying a set of instructions for execution by the machine and that cause the machine to perform any one or more of the methodologies of the various implementations, or that is capable of storing, encoding, or carrying data structures utilized by or associated with such a set of instructions. The term “machine-readable medium” can accordingly be taken to include, but not be limited to, solid-state memories, optical media, and magnetic media. A variety of different types of memory storage devices, such as a random access memory (RAM) or a read only memory (ROM) in the system or a floppy disk, hard disk, CD ROM, DVD ROM, flash, or other computer readable medium that is read from and/or written to by a magnetic, optical, or other reading and/or writing system that is coupled to the processing device, can be used for the memory or memories.

### Introduction

Pancreatic Ductal Adenocarcinoma (PDAC), a common type of Pancreatic Cancer, is one of the leading causes of cancer-related deaths in both men and women in the United States. The 5-year survival rate for PDAC is only about 7-8%. However, with early-stage diagnosis, the 5-year survival rate can be as high as 20%. Therefore, identification of individuals at high risk allows for follow-up imaging examinations and/or biopsy on these individuals, which may lead to early detection and surgical intervention when the tumors are still resectable and not metastatic.

Conventional methods of PDAC prediction were manual, weak, and/or relied mainly on clinical indicators (such as pancreatic duct dilation, pancreatic duct tortuosity, etc.). Most of the existing methods were developed for detection or diagnosis of PDAC, but not for prediction of PDAC, nor diagnosis of very early PDAC. Such disadvantages of the existing methods were, in part, caused by the unavailability of reliable and sufficient predictors. In addition, most of the existing methods are based only on clinical indicators.

Imaging provides a unique opportunity to examine the anatomical and textural changes in the pancreas during the development of PDAC. Such changes are difficult to be examined by naked eyes. Therefore, according to some implementations of the present disclosure, medical imaging such as Computed Tomography (CT) can play an essential role in prediction of PDAC, for example, by allowing a comprehensive evaluation of the morphological and/or textural changes in the pancreas during or before the development of PDAC. To solve the problems of the existing methods and address other needs, systems and methods are disclosed herein that use radiomics-based machine learning algorithm to examine and/or evaluate features in CT images of pancreas, which aids in predicting PDAC and/or identifying PDAC at an early stage.

According to some implementations of the present disclosure, a machine learning prediction model for PDAC is disclosed to identify individuals who have a high risk for PDAC in the near future (e.g., about 6 months to 3 years). In some implementations, the machine learning prediction model utilizes radiomics analysis of CT images of pancreas. The CT images have unique patterns (e.g., anatomical, textural, etc.) in healthy pancreas, cancerous pancreas, and high-risk pancreas (e.g., when pancreas is likely to develop cancer in the near future), that can be revealed using radiomics.

Using the example systems and methodologies disclosed herein, twenty-eight (28) cases for each of the following groups were evaluated: (1) retrospective CT images with PDAC diagnosis, referred as “Cancerous”; (2) retrospective CT images up to three years before PDAC diagnosis that were deemed “normal” by radiologists, referred as “Pre-diagnosed” or “High-risk”; (3) retrospective CT images of a group of subjects who underwent similar imaging studies for reasons unrelated to pancreatic disorders, referred as “Control” or “Healthy.” In some implementations, the “High-risk” group includes individuals who have shown indicators (e.g., (features) of PDAC before conventionally diagnosable PDAC develops. These indicators have different range of values for different groups (e.g., “Healthy,” “Cancerous”, “High-risk”). Examples ranges of the indicators are described below in Table 1.

As evidence by the test data disclosed herein, radiomics analysis of pancreatic CT images can assist in predicting pancreatic cancer up to three years prior to the cancer development. Further, a Naïve Bayes classifier can be trained and tested, using a four-fold cross validation process. The model is at least 80% accurate in predicting PDAC on CT scans of pancreas that appears “normal/healthy” to the naked eye. Thus, the disclosed systems and methods can be used by medical professionals as an additional support when examining CT images of pancreas for predicting PDAC and/or identifying very early stages of PDAC.

### Example Systems and Methodologies

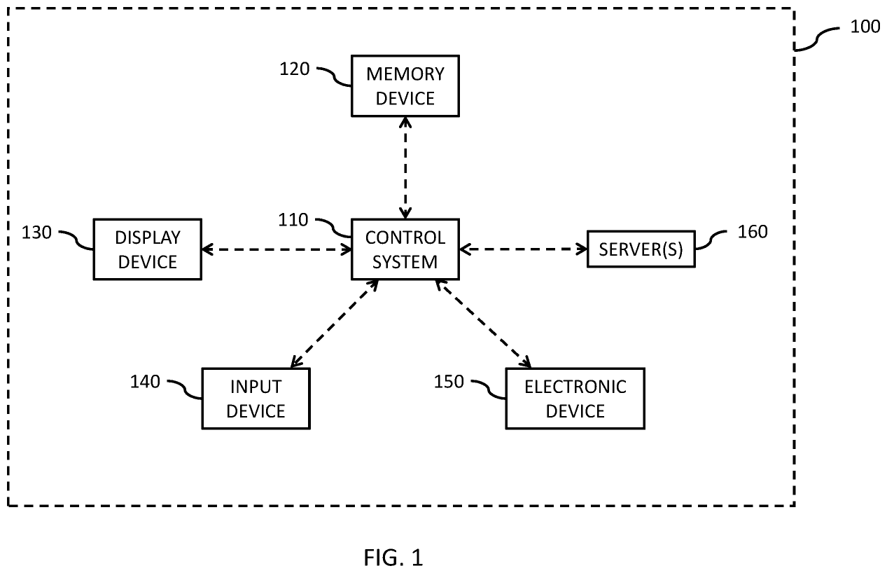

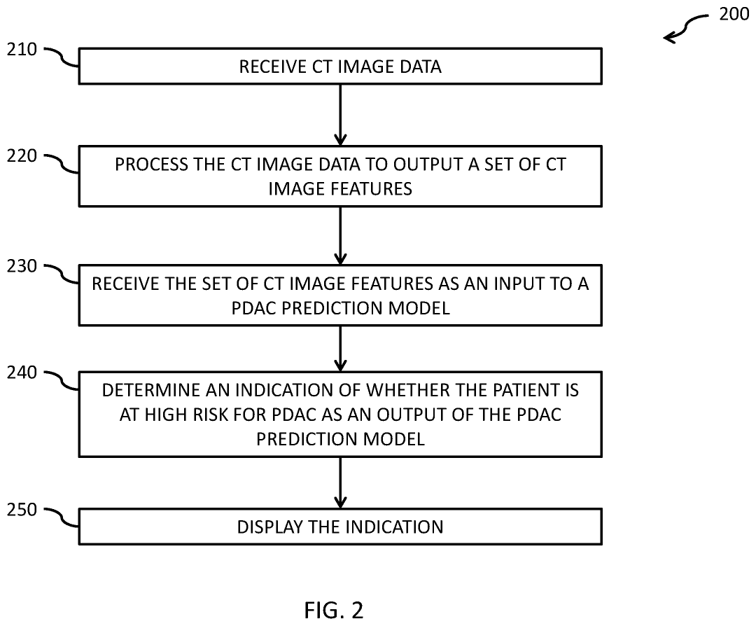

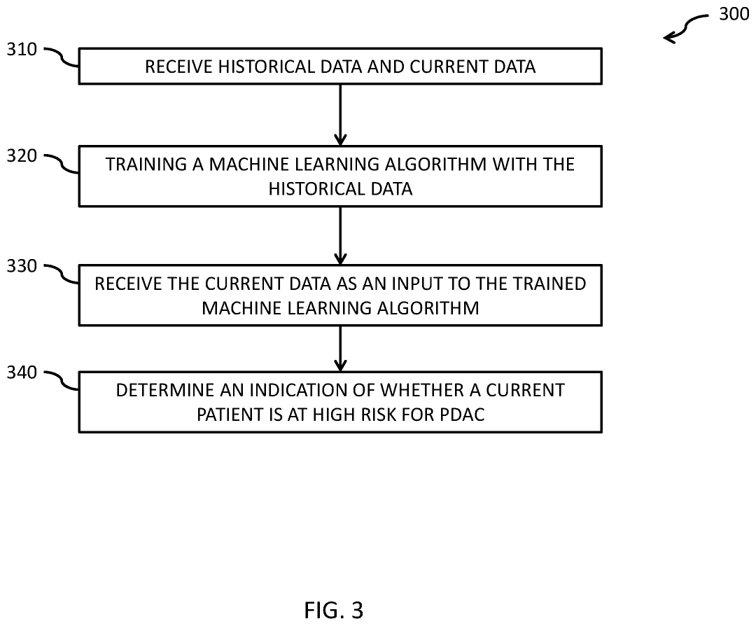

The present disclosure contemplates that a variety of systems can be used to perform various embodiments of the present disclosure. Referring now to FIG. 1, a functional block diagram of a system for identifying individuals at risk for PDAC is shown, according to some implementations of the present disclosure. The system 100 can be configured to perform various methods of the present disclosure, including methods 200 and 300 of FIGS. 2 and 3, respectively.

As depicted in FIG. 1, a system 100 includes a control system 110, a memory device 120, a display device 130, and an input device 140. In some implementations, the system 100 also includes an electronic device 150 for generating image data (e.g., a CT scanner). In some implementations, the system 100 further includes one or more servers 160.

The system 100 generally can be used to generate and/or receive a set of device data (e.g., CT image data) associated with a user (e.g., an individual, a person, a patient, etc.) of the electronic device 150. Alternatively or additionally, the system 100 can be used to generate and/or receive a set of clinical data associated with the patient. For example, in some implementations, the set of clinical data can include medical records data (e.g., diagnosis data). The generated and/or received sets of data, in turn, can be analyzed by the system 100 (e.g., using one or more trained algorithms) to predict whether the patient is at high risk for PDAC.

The control system 110 includes one or more processors. As such, the control system 110 can include any suitable number of processors (e.g., one processor, two processors, five processors, ten processors, etc.). In some implementations, the control system 110 includes one or more processors, one or more memory devices (e.g., the memory device 120, or a different memory device), one or more electronic components (e.g., one or more electronic chips or components, one or more printed circuit boards, one or more power units, one or more graphical processing units, one or more input devices, one or more output devices, one or more secondary storage devices, one or more primary storage devices, etc.), or any combination thereof. In some implementations, the control system 110 includes the memory device 120 or a different memory device, yet in other implementations, the memory device 120 is separate and distinct from the control system 110, but in communication with the control system 110.

The control system 110 generally controls (e.g., actuate) the various components of the system 100 and/or analyzes data obtained and/or generated by the components of the system 100. For example, the control system 110 is arranged to provide control signals to the display device 130, the input device 140, the electronic device 150, or any combination thereof. The control system 110 executes machine readable instructions that are stored in the memory device 120 or a different memory device. The one or more processors of the control system 110 can be general or special purpose processors and/or microprocessors.

While the control system 110 is described and depicted in FIG. 1 as being a separate and distinct component of the system 100, in some implementations, the control system 110 is integrated in and/or directly coupled to the to the display device 130, the input device 140, and/or the electronic device 150. The control system 110 can be coupled to and/or positioned within a housing of to the display device 130, the input device 140, the electronic device 150, or any combination thereof. The control system 110 can be centralized (within one housing) or decentralized (within two or more physically distinct housings).

While the system 100 is shown as including a single memory device 120, it is contemplated that the system 100 can include any suitable number of memory devices (e.g., one memory device, two memory devices, five memory devices, ten memory devices, etc.). The memory device 120 can be any suitable computer readable storage device or media, such as, for example, a random or serial access memory device, a hard drive, a solid state drive, a flash memory device, etc. The memory device 120 can be coupled to and/or positioned within a housing of the to the display device 130, the input device 140, the electronic device 150, the control system 110, or any combination thereof. The memory device 120 can be centralized (within one housing) or decentralized (within two or more physically distinct housings).]

The display device 130 of the system 100 is generally used to display text(s) and/or image(s). The image(s) can include still images, video images, projected images, holograms, or the like, or any combination thereof; and/or information regarding to the display device 130, the input device 140, the electronic device 150, or any combination thereof. For example, the display device 130 can provide information regarding the status of the to the display device 130, the input device 140, the electronic device 150 (e.g., the CT scanner), and/or other information. In some implementations, the display device 130 is included in and/or is a portion of the CT scanner. In some implementations, the display device 130 is included in and/or is a portion of the input device 140.

The display device 130 is configured to receive data from the control system 110, and/or the input device 140, and/or the electronic device 150, and/or the server 160. In some implementations, the display device 130 displays input received from the input device 140. In some implementations, data is first sent to the control system 110, which then processes the data and instructs the display device 130 according to the processed data. In some implementations, the display device 130 displays data directly received from the control system 110. In some implementations, the display device 130 displays the texts(s) and/or image(s), and relays the data to the control system 110. In some implementations, the data is then stored in the memory device 120. Examples of such data include a patient profile, CT images, CT image features, a diagnosis prediction, historical medical data, current medical data, or any combination thereof.

The present disclosure also contemplates that more than one display 130 can be used in system 100, as would be readily contemplated by a person skilled in the art. For example, one display can be viewable by a patient, while additional displays are visible to researchers and/or medical professionals and not to the patient. The multiple displays can output identical or different information, according to instructions by the control system 110.

The input device 140 of the system 100 is generally used to receive user input to enable user interaction with the control system 110, the memory 114, the display device 130, the electronic device 150, or any combination thereof. The input device 140 can include a microphone for speech, a touch-sensitive screen for gesture or graphical input, a keyboard, a mouse, a motion input, or any combination thereof. In some instances, the input device 140 includes multimodal systems that enable a user to provide multiple types of input to communicate with the system 100. The input device 140 can alternatively or additionally include a button, a switch, a dial to allow the user to interact with the system 100. The button, the switch, or the dial may be a physical structure, or a software application accessible via the touch-sensitive screen. In some implementations, the input device 140 may be arranged to allow the user to select a value and/or a menu option. In some implementations, the input device 140 is included in and/or is a portion of the CT scanner. In some implementations, the input device 140 is included in and/or is a portion of the display device 130.

In some implementations, the input device 140 includes a processor, a memory, and a display device, that are the same as, or similar to, the processor(s) of the control system 110, the memory device 120, and the display device 130. In some implementations, the processor and the memory of the input device 140 can be used to perform any of the respective functions described herein for the processor and/or the memory device 120. In some implementations, the control system 110 and/or the memory 114 is integrated in the input device 140.

The display device 130 alternatively or additionally acts as a human-machine interface (HMI) that includes a graphic user interface (GUI) configured to display the image(s) and an input interface. The display device 130 can be an LED display, an OLED display, an LCD display, or the like. The input interface can be, for example, a touchscreen or touch-sensitive substrate, a mouse, a keyboard, or any sensor system configured to sense inputs made by a human user interacting with the system 100 with or without direct user contact/touch.

While the display device 130 and the input device 140 is described and depicted in FIG. 1 as being separate and distinct components of the system 100, in some implementations, the display device 130 and/or the input device 140 are integrated in and/or directly coupled to one or more of the electronic device 150, and/or the control system 110, and/or the memory 120.

The control system 110 can be communicatively coupled to the memory device 120, the display 130, the input device 140, and the electronic device 150. Further, the control system 110 can be communicatively coupled to the server 160. For example, the communication can be wired or wireless. The control system 110 is configured to perform any methods as contemplated according to FIGS. 2-3 (discussed further herein). The control system 110 can process and/or store input from the memory device 120, the display 130, the input device 140, and the electronic device 150. In some implementations, the methodologies disclosed herein can be implemented, via the control system 110, on the server 160. It is also contemplated that the server 160 includes a plurality of servers, and can be remote or local. Optionally, the control system 110 and/or the memory device 120 may be incorporated into the server 160.

While the system 100 is shown as including all of the components described herein with respect to FIG. 1, more or fewer components can be included in a system for generating CT image data, analyzing the CT image data using a trained algorithm, and in turn, predicting whether the patient is at high risk of PDAC. For example, a first alternative system includes the control system 110, the memory 120, and the electronic device 150. As another example, a second alternative system includes the control system 110, the electronic device 150, and the server 160. As yet another example, a third alternative system includes the control system 110, the memory 120, the display device 130, and the input device 140. Thus, various systems for identifying individuals at risk for PDAC can be formed using any portion or portions of the components shown and described herein and/or in combination with one or more other components.

Turning now to FIG. 2, a method 200 for identifying individuals at risk for PDAC is illustrated, according to some implementations of the present disclosure. At step 210, CT image data associated with a pancreas of a patient is received, via, for example, a control system. Alternatively or additionally, CT image data associated with a pancreas of a patient is generated using a CT scanner.

At step 220, The CT image data is processed, using one or more processors, to output a set of CT image features. In some implementations, the set of CT image features is indicative of a variation in morphology of the pancreas (e.g., a size, a shape, a signal intensity, or any combination thereof). In some such implementations, each of the size, shape, and signal intensity is a base class that consists of a plurality of features. For example, there may be hundreds of features that can be extracted on the signal intensity class. Alternatively or additionally, the set of CT image features is indicative of a change in texture of the pancreas (e.g., tissue heterogeneity, run length non-uniformity, inverse autocorrelation, long run emphasis, and short run emphasis, or any combination thereof). Example ranges of values for the CT image features are shown below in Table 1.

TABLE 1
ranges of values for different groups of individuals

| | Tissue | Run length | Inverse auto | Long run | Short run |
|---|---|---|---|---|---|
| | Heterogeneity | non-uniformity | correlation | emphasis | emphasis |
| Healthy | Mean = 0.4110 | Mean = 0.4127 | Mean = 0.5747 | Mean = 0.4269 | Mean = 0.4269 |
| | S.D = 0.2610 | S.D. = 0.2487 | S.D. = 0.2426 | S.D. = 0.2594 | S.D. = 0.2594 |
| High-risk | Mean = 0.2916 | Mean = 0.2822 | Mean = 0.7143 | Mean = 0.2868 | Mean = 0.7127 |
| | S.D = 0.2797 | S.D. = 0.2680 | S.D. = 0.2652 | S.D. = 0.2740 | S.D. = 0.2693 |
| Diagnosed | Mean = 0.1734 | Mean = 0.1622 | Mean = 0.8245 | Mean = 0.1366 | Mean = 0.8311 |
| | S.D = 0.2511 | S.D. = 0.2192 | S.D = 0.30 | S.D. = 0.2842 | S.D. = 0.1934 |

At step 230, the set of CT image features is received as an input to a PDAC prediction model. At step 240, an indication of whether the patient is at high risk for PDAC is determined as an output of the PDAC prediction model. At step 250, the indication is displayed on a display device. In some implementations, the determining the indication of whether the patient is at high risk for PDAC includes determining whether the set of CT image features is indicative of pre-cancerous tissue changes in the pancreas of the patient. In some implementations, the PDAC prediction model is a machine learning algorithm. In some implementations, the machine learning PDAC prediction algorithm includes a K-means clustering, a Logistic Regression, a Support Vector Machine, a Naïve Bayes classifier, a Nearest Neighbors, or any combination thereof.

The machine learning PDAC prediction algorithm can be trained with historical data for historical patients. In some implementations, the historical data includes a plurality of CT image features of a pancreas and a corresponding PDAC diagnosis (e.g., healthy, pre-cancerous, cancerous) of each of the historical patients. The plurality of CT image features can be extracted from retrospective CT images of the pancreas of the each of the historical patients.

Referring now to FIG. 3, a method 300 for identifying individuals at risk for PDAC using machine learning is illustrated, according to some implementations of the present disclosure. At step 310, data associated with a plurality of individuals is received. The data includes historical data of historical patients and current data of a current patient.

In some implementations, the historical data includes retrospective CT images of a pancreas and a corresponding PDAC diagnosis of each of the historical patients. In some implementations, the historical data includes a plurality of CT image features of a pancreas and a corresponding PDAC diagnosis of each of the historical patients. For example, the plurality of CT image features can be extracted from retrospective CT images of the pancreas of the each of the historical patients.

In some implementations, the plurality of CT image features of the historical data is indicative of a variation in morphology of the pancreas. For example, the morphology can include a size, a shape, a signal intensity, or any combination thereof. In some implementations, the plurality of CT image features of the historical data is indicative of a change in texture of the pancreas. For example, the change in texture can be tissue heterogeneity, run length non-uniformity, inverse autocorrelation, long run emphasis, and short run emphasis.

In some implementations, the current data of the current patient includes a set of CT image features associated with CT images of a pancreas of the current patient. For example, the set of CT image features associated with the CT images of the pancreas of the current patient can be extracted from the CT images of the pancreas of the current patient.

At step 320, a machine learning algorithm is trained with the historical data, using, for example, K-means clustering, Logistic Regression, Support Vector Machine, Naïve Bayes classifier, Nearest Neighbors, or any combination models thereof. At step 330, the current data of the current patient is received as an input to the trained machine learning algorithm. At step 340, an indication of whether the current patient is at high risk for PDAC is determined as an output of the trained machine learning algorithm.

### Example Application of the Disclosed Models

According to some implementations of the present disclosure, an automated prediction model is developed to identify individuals at high risk for PDAC in the near future using radiomic analysis of their CT scans of pancreas. The radiomics analysis allows identification of image features, such as variations in morphology (size, shape, signal intensity) and texture, associated with pre-cancerous tissue changes in CT pancreatic images. Twenty-eight (28) retrospective CT scans of pancreas were obtained, from each of three groups as: (1) Diagnosed: scans with established PDAC (observable tumor); (2) Pre-cancerous/High-risk: scans of same subjects (of Diagnosed category), obtained up to three years before their PDAC diagnosis that were deemed “normal” by radiologists, and (3) Healthy Control: abdominal scans with no pancreatic disorders.

Up to 5,000 quantifiable radiomic quantities (using different radiomics parameters) were extracted from the manually segmented pancreas in CT scans of three groups. From this set, features were identified, predictive of PDAC, which are significantly different in three groups (identified through, for example, statistical significance t-test). In addition, the identified features demonstrate a linear incremental or decremental trend from time order prospective of three groups.

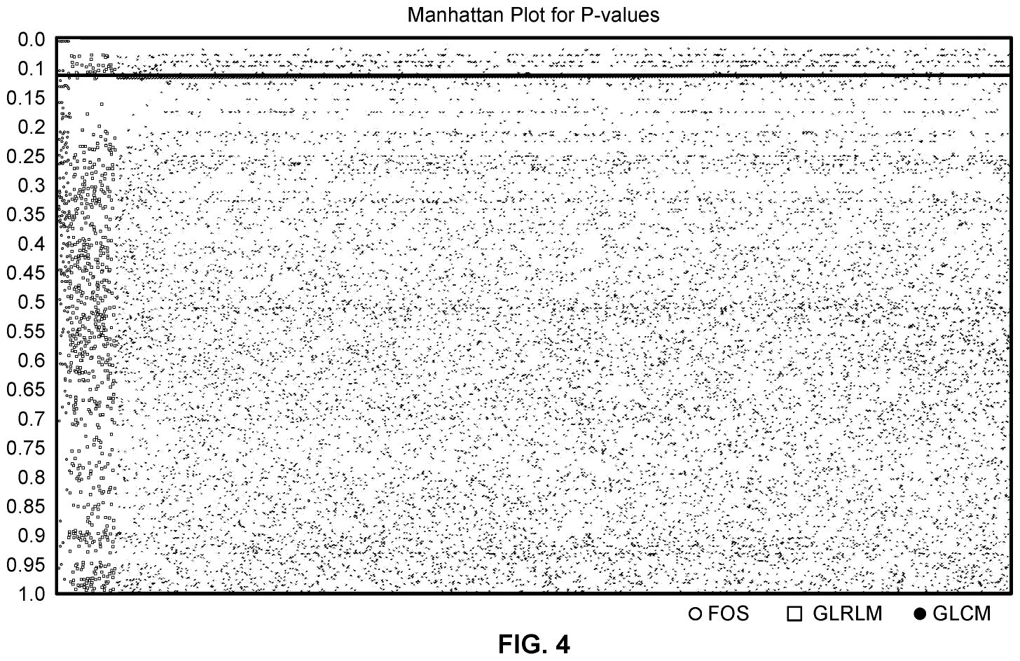

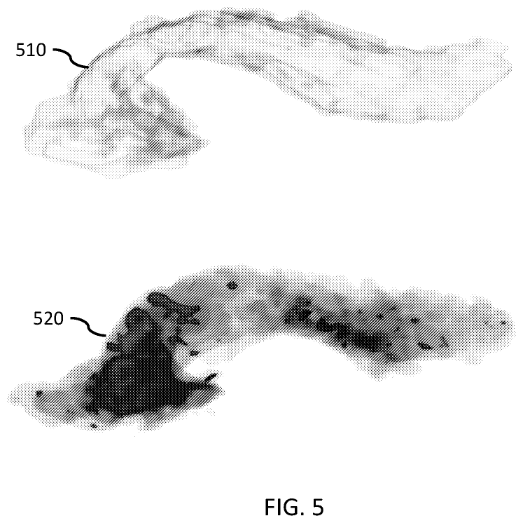

FIGS. 4-5 show evaluations using the disclosed methods (discussed further with regard to FIGS. 2-3 and corresponding description). Nearly seven percent of total radiomic features were found potentially predictive of PDAC, as shown in FIG. 4, which illustrates a Manhattan plot of the predictive features over horizontal line at p-value=0.05. Units on the y-axis are p-values. There no units on the X-axis, but three types of features: FOS stands for first order statistics; GLRLM stands for grey-level run-length matrix; and GLCM stands for grey-level c-occurrence matrix. As shown, features above the horizontal line in FIG. 4 are significantly different between groups, and thus can be used as predictive features, some of which are included herein as features indicative of early stage PDAC, such as the features listed in Table 1.

To develop the PDAC prediction model with a few best features, Recursive Feature Elimination (RFE) method was applied for five machine learning methods (e.g., K-means clustering, Logistic Regression, Support Vector Machine, Naïve Bayes classifier, and Nearest Neighbors) to (1) recursively removed the weakest predictive features during training of all of these methods, and (2) identify the one that uses the least number of predictive features to get highest prediction accuracy.

With four-fold cross-validation, Naïve Bayes classifier produced eighty (80) percent accuracy (highest among all methods) for identifying CT scans with high-risk for PDAC, out of the total 84 CT scans (28 each from the three categories). The classifier was trained on five best features, including tissue heterogeneity, run length non-uniformity, inverse autocorrelation, long run emphasis, and short run emphasis. Results processed using the five best features are tested and verified in FIG. 5, which is a combined feature map of the five features (e.g., predictors) for the healthy pancreas 510 and for the pre-cancerous pancreas 520. The feature map in FIG. 5 shows the textural changes for the pre-cancerous pancreas 520, which is predictive of PDAC.

Thus, the CT scans of pre-cancerous pancreas show unique features that can assist in the prediction of PDAC. The developed prediction models of the present disclosure aid in identifying such pre-cancerous and/or high-risk pancreas. In addition, a large dataset can be utilized (e.g., at one or more steps of the method 300) to further validate the disclosed models. Moreover, deep learning techniques may be applied to uncover other complex predictors in pre-cancerous CT scans of pancreas.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present disclosure, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or additionally, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### CONCLUSION

One or more elements or aspects or steps, or any portion(s) thereof, from one or more of any of claims 1-30 below can be combined with one or more elements or aspects or steps, or any portion(s) thereof, from one or more of any of the other claims 1-30 or combinations thereof, to form one or more additional implementations and/or claims of the present disclosure.

While various examples of the present disclosure have been described above, it should be understood that they have been presented by way of example only, and not limitation. Numerous changes to the disclosed examples can be made in accordance with the disclosure herein without departing from the spirit or scope of the disclosure. Thus, the breadth and scope of the present disclosure should not be limited by any of the above described examples. Rather, the scope of the disclosure should be defined in accordance with the following claims and their equivalents.

Although the disclosure has been illustrated and described with respect to one or more implementations, equivalent alterations and modifications will occur to others skilled in the art upon the reading and understanding of this specification and the annexed drawings. In addition, while a particular feature of the disclosure may have been disclosed with respect to only one of several implementations, such feature may be combined with one or more other features of the other implementations as may be desired and advantageous for any given or particular application.

The terminology used herein is for the purpose of describing particular examples only and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this disclosure belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

1. A system for identifying individuals at risk for pancreatic ductal adenocarcinoma (PDAC), the system comprising:
a CT scanner configured to generate CT image data associated with a pancreas of a patient;
a memory storing machine-readable instructions; and
a control system including one or more processors configured to execute the machine-readable instructions to:
receive the CT image data associated with the pancreas of the patient;
process the received CT image data to output a set of CT image features;
receive, as an input to a machine learning PDAC prediction algorithm, the set of CT image features; and
determine, as an output of the machine learning PDAC prediction algorithm, an indication of whether the patient is at high risk for PDAC.

2. The system of claim 1, wherein the control system including the one or more processors is further configured to execute the machine-readable instructions to display, on a display device of the system, the indication of whether the patient is at high risk for PDAC.

3. The system of claim 1, wherein the control system including the one or more processors is further configured to execute the machine-readable instructions to train the machine learning PDAC prediction algorithm with historical data for historical patients, the historical data including a plurality of CT image features of a pancreas and a corresponding PDAC diagnosis of each of the historical patients, the plurality of CT image features being extracted from retrospective CT images of the pancreas of the each of the historical patients.

4. The system of claim 3, wherein the PDAC diagnosis is healthy, pre-cancerous, or cancerous.

5. The system of claim 1, wherein the set of CT image features is indicative of a variation in morphology of the pancreas.

6. The system of claim 5, wherein the morphology includes a size, a shape, a signal intensity, or any combination thereof.

7. The system of claim 1, wherein the set of CT image features is indicative of a change in texture of the pancreas.

8. The system of claim 1, wherein the set of CT image features includes at least one of tissue heterogeneity, run length non-uniformity, inverse autocorrelation, long run emphasis, and short run emphasis.

9. The system of claim 1, wherein the machine learning PDAC prediction algorithm includes a K-means clustering, a Logistic Regression, a Support Vector Machine, a Naïve Bayes classifier, a Nearest Neighbors, or any combination thereof.

10. The system of claim 1, wherein the machine learning PDAC prediction algorithm includes a Naïve Bayes classifier.

11. A method for identifying individuals at risk for pancreatic ductal adenocarcinoma (PDAC), using machine learning, the method comprising:
receiving data associated with a plurality of individuals, the data including historical data of historical patients and current data of a current patient, the current data including a set of CT image features associated with CT images of a pancreas of the current patient; and
training a machine learning algorithm with the historical data such that the machine learning algorithm is configured to:
receive, as an input, the current data of the current patient, and
determine, as an output, an indication of whether the current patient is at high risk for PDAC.

12. The method of claim 11, wherein the historical data includes retrospective CT images of a pancreas and a corresponding PDAC diagnosis of each of the historical patients.

13. The method of claim 12, wherein the PDAC diagnosis is healthy, pre-cancerous, or cancerous.

14. The method of claim 11, wherein the historical data includes a plurality of CT image features of a pancreas and a corresponding PDAC diagnosis of each of the historical patients, the plurality of CT image features being extracted from retrospective CT images of the pancreas of the each of the historical patients.

15. The method of claim 14, wherein the set of CT image features associated with the CT images of the pancreas of the current patient is extracted from the CT images of the pancreas of the current patient.

16. The method of claim 14, wherein the plurality of CT image features of the historical data is indicative of a variation in morphology of the pancreas.

17. The method of claim 16, wherein the morphology includes a size, a shape, a signal intensity, or any combination thereof.

18. The method of claim 14, wherein the plurality of CT image features of the historical data is indicative of a change in texture of the pancreas.

19. The method of claim 14, wherein the plurality of CT image features of the historical data includes at least one of tissue heterogeneity, run length non-uniformity, inverse autocorrelation, long run emphasis, and short run emphasis.

20. The method of claim 11, wherein the machine learning algorithm includes a K-means clustering, a Logistic Regression, a Support Vector Machine, a Naïve Bayes classifier, a Nearest Neighbors, or any combination thereof.

21. The method of claim 11, wherein the machine learning algorithm includes a Naïve Bayes classifier.

22. A method for identifying individuals at risk for pancreatic ductal adenocarcinoma (PDAC), the method comprising:
generating, using a CT scanner, CT image data associated with a pancreas of a patient;
processing, using one or more processors, the CT image data to output a set of CT image features;
receiving, as an input to a PDAC prediction model, the set of CT image features;
determining, as an output of the PDAC prediction model, an indication of whether the patient is at high risk for PDAC; and
displaying, on a display device, the indication.

23. The method of claim 22, wherein the set of CT image features is indicative of a variation in morphology of the pancreas.

24. The method of claim 23, wherein the morphology includes a size, a shape, a signal intensity, or any combination thereof.

25. The method of claim 22, wherein the set of CT image features is indicative of a change in texture of the pancreas.

26. The method of claim 22, wherein the set of CT image features includes at least one of tissue heterogeneity, run length non-uniformity, inverse autocorrelation, long run emphasis, and short run emphasis.

27. The method of claim 22, wherein the PDAC prediction model includes a K-means clustering, a Logistic Regression, a Support Vector Machine, a Naïve Bayes classifier, a Nearest Neighbors, or any combination thereof.

28. The method of claim 22, wherein the PDAC prediction model includes a Naïve Bayes classifier.

29. The method of claim 22, wherein the indication is that the patient is healthy, the patient is pre-cancerous, or the patient is cancerous.

30. The method of claim 22, wherein the determining the indication of whether the patient is at high risk for PDAC includes determining whether the set of CT image features is indicative of pre-cancerous tissue changes in the pancreas of the patient.
